(19) United States
(12) Patent Application Publication
(10) Pub. No.: US 2009/0264686 A1
(43) Pub. Date: Oct. 22, 2009

(54) Hydrogenolysis processes and hydrogenolysis catalyst preparation methods

(76) Inventors: Johnathan E. Holladay, Kennewick, WA (US); James F. White, Richland, WA (US); Thomas H. Peterson, Midland, MI (US); John G. Frye, JR., Richland, WA (US); Danielle S. Muzatko, Kennewick, WA (US); Simon R. Bare, Wheaton, IL (US); James G. Vassilakis, Naperville, IL (US); Richard R. Rosin, Glencoe, IL (US)

Correspondence Address: WELLS ST. JOHN P.S., 601 WEST FIRST AVENUE, SUITE 1300, SPOKANE, WA, 99201-3828, US

(21) Appl. No.: 12/082,997

(22) Filed: Apr. 16, 2008

(51) Int. Cl. B01J 23/44; C07C 29/60; B01J 23/755; B01J 23/75

(52) U.S. Cl. 568/861; 502/439; 502/337; 502/300; 502/326

(57) ABSTRACT

Hydrogenolysis processes are provided that can include providing a hydrogenolysis reactor having a catalyst therein. The catalyst can be exposed to a reducing agent in the absence of polyhydric alcohol compound while maintaining a temperature of the catalyst above 290° C. Hydrogenolysis processes can also include providing a passivated catalyst to within a reactor and exposing the catalyst to a reducing atmosphere while maintaining the catalyst at a temperature less than 210° C.

Hydrogenolysis catalyst preparation methods are provided that can include exposing the catalyst to a first reducing atmosphere while maintaining the catalyst at a first temperature to reduce at least a portion of the catalyst. The method can also include passivating at least the portion of the catalyst and depassivating the portion of the catalyst in the presence of a second reducing atmosphere while maintaining the portion of the catalyst at a second temperature less than the first temperature.

### TECHNICAL FIELD

The present disclosure relates to hydrogenolysis processes and hydrogenolysis catalyst preparation methods.

### BACKGROUND

By-product compounds have been identified during the production of fuel from organic material such as the production of biodiesel from plant material. Many of these by-products are of low commercial value but with modification can be of high commercial value. One such compound is glycerol, which is a by-product from the biodiesel manufacturing process. Hydrogenolysis of glycerol to yield relatively more commercially valuable compounds such as propylene glycol can be performed. The conversion of multihydric alcohol compounds such as glycerol to polyols such as propylene glycol can be beneficial for at least the reason that substantial waste by-products of biodiesel manufacturing process can be eliminated. The present disclosure provides methods for increasing the efficiency of these types of hydrogenolysis processes and in particular embodiments, discloses hydrogenolysis catalyst preparation methods.

### SUMMARY OF THE DISCLOSURE

Hydrogenolysis processes are provided that can include providing a hydrogenolysis reactor having a catalyst therein. The catalyst can include Re and one or both of Co and Pd. The catalyst can be exposed to a reducing agent in the absence of polyhydric alcohol compound while maintaining a temperature of the catalyst above 290° C. The process can also include contacting the catalyst with the polyhydric alcohol compound.

Hydrogenolysis processes can also include providing a passivated catalyst to within a reactor and exposing the catalyst to a reducing atmosphere while maintaining the catalyst at a temperature less than 210° C. The process may continue by contacting the catalyst with the polyhydric alcohol compound.

Hydrogenolysis catalyst preparation methods are provided that can include exposing the catalyst to a first reducing atmosphere while maintaining the catalyst at a first temperature to reduce at least a portion of the catalyst. The first temperature can be the greatest temperature of the catalyst during the exposing. The method can also include passivating at least the portion of the catalyst and depassivating the portion of the catalyst in the presence of a second reducing atmosphere while maintaining the portion of the catalyst at a second temperature less than the first temperature.

Preparation methods can also include providing a hydrogenolysis catalyst and maintaining the catalyst at a temperature of at least about 280° C. in the presence of a continuous supply of inert atmosphere.

### BRIEF DESCRIPTION OF THE DRAWINGS

Preferred embodiments of the disclosure are described below with reference to the following accompanying drawings.

FIG. 1 is a catalyst preparation system according to an embodiment.

FIG. 2 is graphical representation of data acquired utilizing the processes and methods described according to an embodiment.

FIG. 3 is graphical representation of data acquired utilizing the processes and methods described according to an embodiment.

FIG. 4 is graphical representation of data acquired utilizing the processes and methods described according to an embodiment.

FIG. 5 is graphical representation of data acquired utilizing the processes and methods described according to an embodiment.

FIG. 6 is graphical representation of data acquired utilizing the processes and methods described according to an embodiment.

FIG. 7 is graphical representation of data acquired utilizing the processes and methods described according to an embodiment.

FIG. 8 is graphical representation of data acquired utilizing the processes and methods described according to an embodiment.

FIG. 9 is graphical representation of data acquired utilizing the processes and methods described according to an embodiment.

FIG. 10 is graphical representation of data acquired utilizing the processes and methods described according to an embodiment.

FIG. 11 is graphical representation of data acquired utilizing the processes and methods described according to an embodiment.

### DESCRIPTION

This disclosure is submitted in furtherance of the constitutional purposes of the U.S. Patent Laws “to promote the progress of science and useful arts” (Article 1, Section 8).

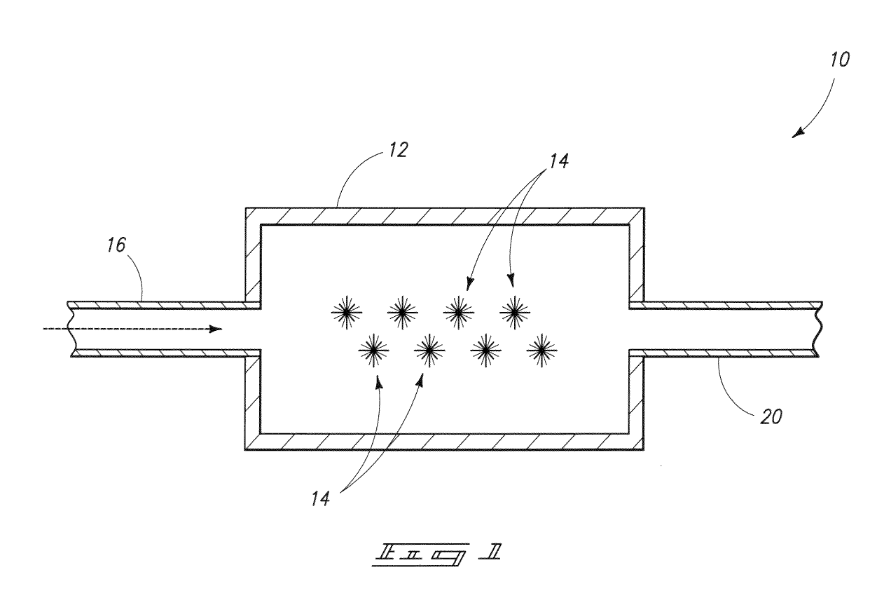

Hydrogenolysis processes and hydrogenolysis catalyst preparation methods are described with reference to FIGS. 1-11. Referring first to FIG. 1, a chemical production process system 10 is shown that includes a reservoir 12 housing catalyst 14. Reservoir 12 is in fluid communication with conduit 16 and conduit 20.

According to example embodiments, reservoir 12 can be configured with additional conduits (not shown), for example to provide a reactant mixture thereto. According to example implementations, reservoir 12 can be a chamber that is configured to house catalyst as well as have the temperature and pressure of the interior of the chamber be maintained throughout a range of temperatures and pressures. Reservoir 12 can also be configured as a reactor and as such, the reactor can be any reactor suitable for use under desired conditions of temperature, pressure, solvent, and/or contact time. Examples of suitable chambers include but are not limited to: trickle bed, bubble column reactors, and continuous stirred tanks, for example. Reservoir 12 can be used in-line in chemical processes and can be effectively coupled with various additional components of chemical production processes such as cation exchange columns, distillation columns, etc., and can be used in various embodiments of the present disclosure. The flow of materials such as reactants and/or reducing atmospheres through reservoir 12 can be manipulated with flow controllers and/or pressure differentiation apparatuses, for example.

Catalyst 14 can be multi-metallic catalysts such as bi or tri metallic catalysts. According to example embodiments, catalyst 14 can comprise one or both of Ni and Re. Via conduit 16, catalyst 14 can be exposed to a reducing agent. Example reducing agents include H₂. Catalyst 14 can be exposed to this reducing agent in the absence of polyhydric alcohol reactants such as polyhydric alcohol compounds. According to example implementations, the catalyst can be exposed to this reducing agent while maintaining a temperature of the catalyst within reservoir 12 below about 350° C. Where the catalyst comprises Ni and/or Re, the temperature of the catalyst can be maintained below 290° C. during the exposing. According to example implementations, the catalyst can comprise at least about 5% (wt./wt.) Ni.

The remainder of the catalyst can be provided in a solid form on a support material that is selected to resist degradation under intended reaction conditions, for example. Such support materials are known in the art and may include high surface area oxide supports. Carbon, zirconium and titanium (especially in the rutile form) may be preferred because of their stability in hydrothermal conditions (aqueous solutions at above 100° C. and one atmosphere pressure). Supports can also be formed of mixed or layered materials. For example, in some embodiments, the support can be carbon with a surface layer of zirconia or zirconium mixed with catalyst metals. Of this support material, according to example implementations, 0.7% (wt./wt.) Re may be a part thereof. According to example implementations, the catalyst can include from between about 0.7% (wt./wt.) to about 2.5% (wt./wt.) Re.

According to example embodiments, catalyst preparation can include exposing catalyst 14 to a reducing atmosphere while maintaining the catalyst at a temperature of from between 265° C. and 320° C. The catalyst may then be passivated via exposure to the atmosphere, such exposure taking place, for example, during transfer of catalyst from reduction apparatus to reactor apparatus. Catalyst 14 can then be depassivated in the presence of a reducing agent while maintaining the catalyst at a temperature of less than 320° C. According to example implementatons, where the catalyst comprises one or both of Ni and Re, during the exposing of the catalyst to a reducing atmosphere, the catalyst can be maintained at a temperature of from about 290° C. to about 320° C. The depassivating of the catalyst can include elevating the catalyst temperature from a first temperature to a temperature of less than 320° C. According to example implementations the catalyst can be depassivated by exposing the catalyst to a reducing atmosphere while maintaining the catalyst at a temperature below that which the catalyst was originally reduced at. Elevation can take place at a rate less than about 2° C. per minute and/or at a rate of less than about 1.5° C. per minute. The reducing atmosphere or agent provided during this elevating can include one or both of H₂ and/or N₂. According to example implementations, the reducing agent can be at least about 5% (v/v) H₂, or from about 15% to about 50% H₂, or from about 15% to about 50% H₂.

According to other embodiments, the catalyst can comprise Re and one or both of Co and Pd. As an example, this catalyst can be reduced by exposing the catalyst to a reducing atmosphere while maintaining a temperature of the catalyst above 290° C. or between about 290° C. and about 350° C. or between about 290° C. and about 320° C. The temperature of the catalyst can be maintained for less than about 12 hours or at least 3 hours or from about 3 hours to about 12 hours.

In this catalyst system, the depassivating can include elevating the catalyst temperature from a first temperature to a temperature of less than 210° C. The elevating of this catalyst temperature can include increasing the temperature at a rate of less than 1.5° C. per minute to a temperature less than 210° C. In accordance with one implementation, the catalyst can be reduced at a temperature of at least about 290° C. and depassivated at a temperature of less than about 210° C.

According to example implementations, the exposing of the catalyst to a reducing agent can include elevating the temperature from a first temperature, such as ambient temperature, to at least about 210° C. at a rate of less than about 1.5° C. per minute. According to other implementations, the exposing can include elevating the temperature of the catalyst from a first temperature of at least about 290° C. at a rate of less than about 1.5° C. per minute. The catalyst can be maintained at temperatures from about 265° C. to about 290° C. for hours at a time.

According to other example implementations, a catalyst can comprise one or more of Co, Pd, and Re. Within reservoir 12, this catalyst can be maintained from between about 260° C. and about 350° C. while exposing the catalyst to the reducing agent. The temperature of the catalyst can be also be maintained between about 290° C. and about 350° C. The reducing agent can include both N and H, and the reducing agent can comprise at least about 4% (v/v) H₂.

Catalyst 14 can be a previously activated catalyst that has subsequently become passivated, and this passivated catalyst can be provided to within reservoir 12 acting as a reactor, for example. According to example implementations, the catalyst can be exposed to a reducing agent while maintaining the catalyst at a temperature of less than about 290° C.

In accordance with another example embodiment, hydrogenolysis catalyst can be provided and the catalyst can be maintained at a temperature of at least about 280° C. in the presence of a continuous supply of inert atmosphere such as N₂. The catalyst can comprise Re and one or more of Ni, Co, and Pd. The temperature can maintained for at least about 3 hrs at, for example, 350° C. The inert atmosphere may be continuously supplied at a rate of about 50 ml/hr.

Catalyst of the present processes and preparation can be made by incipient wetness impregnation techniques. A porous support may be purchased or prepared by known methods. A catalytic metal precursor can be prepared or obtained. The precursor may be prepared, for example, by dissolving a metal compound in water or acid or purchasing a precursor in solution. The precursor may be in the form of a cation or an anion. A typical precursor for nickel may be nickel nitrate dissolved in water. A typical precursor for ruthenium may be ruthenium chloride. A typical precursor for rhenium may be perrhenic acid. Each of the precursor materials may be in liquid or solid form; these particles may also contain other components such as halides, cations, anions etc. In some preferred embodiments, organic solvents are avoided and the precursor impregnation solution is prepared only in water. Conditions for preparing precursor solution will depend on the type of metal and available ligands. In the case of a particulate support, such as activated carbon powders, the support and precursor composition can be mixed in a suspension. The porous support is preferably not coated by a vapor-deposited layer, more preferably the method of making the catalyst may not have a vapor deposition step. A catalyst metal can be deposited subsequent to, or simultaneous with, the deposition of a metal oxide. Catalyst metal components can be impregnated into the support in a single-step, or by multi-step impregnation processes. In an example method, the precursor for the catalyst component can be prepared in a single solution that is equivalent in volume to the measured amount of solvent that the porous support will uptake to fill all of the pore volume. This solution can be added to the dry support such that it is absorbed by the support and fills the available pore volume. The support can then be vacuum dried in order to remove the solvent and leave the catalytic metal precursor to coat the surface of the support. Subsequent reduction can reduce the catalytic material to its metallic state or another oxidation state and may disassociate the metal from its anion or cation used to make the metal soluble. In most cases, the catalyst can be reduced prior to use. After subsequent reduction, the catalyst can be exposed to oxygen to be passivated. This passivation is quite common in the art as catalyst is moved between chambers and is exposed to oxygen to thereby passivate the catalyst.

Upon activation and/or depassivation, the catalyst can then be exposed to a polyhydric alcohol compound in the presence of a reducing agent to form a polyol. As an example, the polyhydric alcohol compound can have n hydroxyl groups and the polyol can have n-1 hydroxyl groups. The polyhydric alcohol compound can include n hydroxyl groups, with n being ranging from 2 to 6 hydroxyl groups. The polyhydric alcohol compound can be an oxygen containing organic compound such as a C-3 triol. Example polyhydric alchohol compounds include but are not limited to glycerol. Additional example polyhydric alcohol compounds utilized can be sorbitol.

According to example embodiments, reservoir 12 can be configured has a reactor and conduit 16 can be configured to provide a polyhydric alcohol compound to catalyst 14 within reservoir 12. The polyhydric alcohol compound can be provided to this catalyst in order to hydrogenolyze the polyhydric alcohol compound to form a polyol having one less hydroxyl group. As an example, glycerol can be the polyhydric alcohol compound provided to reservoir 12 having catalyst 14 therein and this polyhydric alcohol compound can contact the catalyst and form propylene glycol, for example. Preparing catalysts as described herein can provide increased efficiency with respect to this hydrogenolysis reaction.

This polyhydric alcohol compound can be an aqueous solution containing as much as 90% water, for example. According to other example implementations, the reactant stream 16 can contain as much as 55% water and/or about 45% polyhydric alcohol compound. This reactant stream may not contain a basic compound according to example implementations.

The pH of reactant stream 16 can be less than 7.0, for example. Reactant stream 16 can constitute the majority of the liquid phase within reactor 12. Reactant stream 16 can also include a reducing agent, for example, H₂. Reactant stream 16 can be in fluid communication with reactor 12, and thereby reactant mixture 16 can be exposed to catalyst 14 within reactor 12. According to example implementations, a mole percent of the reducing agent to the polyhydric compound within reactant stream 16 can be at least about 35% polyhydric compound.

### EXAMPLE 1

### Ni/Re Catalyst Preparation

Two catalysts samples can be prepared using 5% Ni 0.7% Re impregnated on Norit ROX 0.8 carbon extrudate. The samples can be reduced at the following temperatures: 265° C. (catalyst M), 290° C. (catalyst D), 320° C. (catalyst E) under a flow of H₂ and passivated. Each catalyst can be tested individually by loading into a down-flow trickle bed reactor. Catalysts D and E can be activated by raising the temperature of the reactor 2° C./min to 320° C. while flowing a 4% (v/v) H₂ in N₂ mixture at 250 sccm and upon reaching temperature increasing the H₂ concentration to 100% and holding 2 h. The reactor temperature can be lowered to 190° C., the gas flow rate can be increased to 450 sccm and the pressure raised to 1200 psig. Glycerol feed (˜40 wt % glycerol, 2.1 wt % NaOH) can be fed to the reactor at a rate of 1.7 LHSV (40 mL/min).

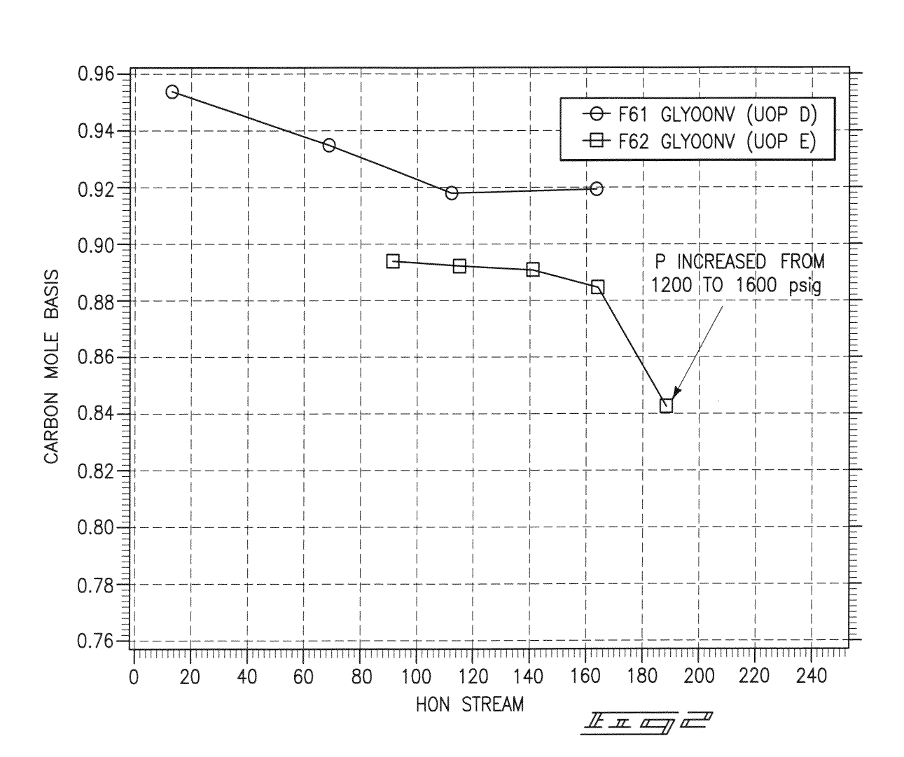

The performance of the two catalysts is shown in FIG. 2. The sample reduced at the lower temperature demonstrated higher activity, as shown by glycerol conversion, than the sample reduced at the higher temperature.

### EXAMPLE 2

### Ni/Re Catalyst Under Batch Conditions

Two catalysts samples can be prepared using 5% Ni 0.7% Re impregnated on Norit ROX 0.8 carbon extrudate. The samples can be reduced at the following temperatures: 265° C. (catalyst M) and 290° C. (catalyst G), under a flow of H₂ and passivated. Each catalyst can be tested individually by loading into a down-flow trickle bed reactor. Catalysts G and M can be activated by raising the temperature of the reactor 1.5° C./min to a desired temperature while flowing H₂ at 250 sccm and holding 2 h. The reactor temperature can be lowered to 190° C., the gas flow rate can be increased to 450 sccm and the pressure raised to 1200 psig. Glycerol feed (˜40 wt % glycerol, 2.1 wt % NaOH) can be fed to the reactor at a rate of 1.7 LHSV (40 mL/min). Two depassivation temperatures can be tested for G, 290 and 210° C. Catalyst M can be depassivated at 210° C. Results are shown in Tables 1 and 2 below.

### EXAMPLE 3

### Co/Pd/Re Catalyst

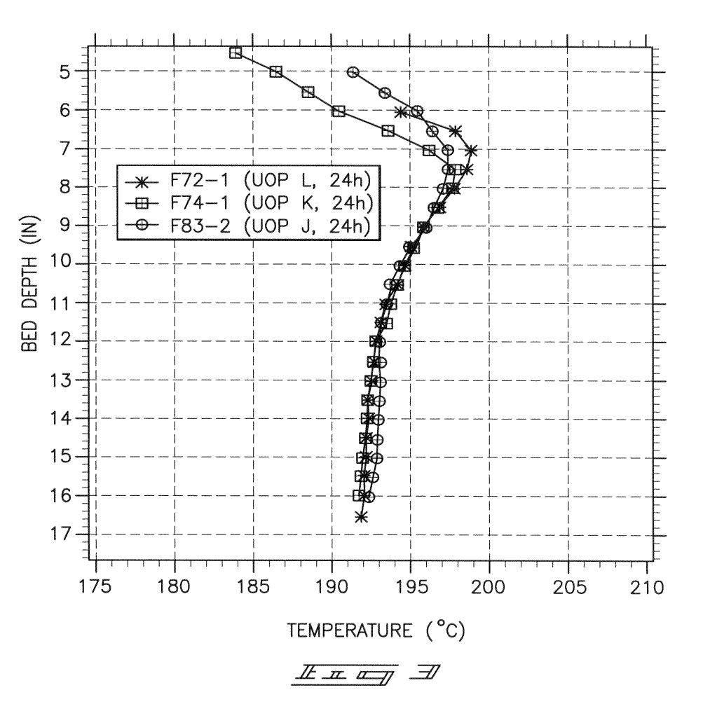

Three catalysts samples can be prepared at a metal loading of 2.5% Co, 0.4% Pd and 2.4% Re on Norit ROX 0.8 extrudate. The catalysts can be reduced at the following temperatures: 260° C. (catalyst J), 290° C. (catalyst K) and 320° C. (catalyst L) for 3 h and passivated. Each catalyst can be tested individually by loading into a down-flow trickle bed reactor. The catalysts can be activated by raising the temperature of the reactor 1.5° C./min to 210° C. while flowing H₂ at 250 sccm and holding 2 h. The reactor temperature can be lowered to 190° C., the gas flow rate can be increased to 450 sccm and the pressure raised to 1200 psig. Glycerol feed (˜40 wt % glycerol, 2.1 wt % NaOH) can be fed to the reactor at a rate of 1.7 LHSV (40 mL/min). Data from the three runs is shown in Table 3 below and depicted graphically in FIG. 3.

TABLE 1
Catalysts M and G.

| | F89-1 | F89-2 | F89-3 | F65-1 | F65-2 | F65-3 | F64-1 | F64-2G | F64-G3 |
|---|---|---|---|---|---|---|---|---|---|
| | Catalyst M | | | Catalyst G | | | Catalyst G | | |
| | Reduction T = 265° C. | | | Reduction T = 290° C. | | | Reduction T = 290° C. | | |
| System Conditions | Depassivation T = 210° C. | | | Depassivation T = 210° C. | | | Depassivation T = 290° C. | | |
| Hours on stream | 24:15:00 | 46:08:00 | 75:40:00 | 23:34:00 | 47:25:00 | 143:14:00 | 70:40:00 | 114:03:00 | 143:36:00 |
| Cat. Bed Temp (° C.) | 190 | 190 | 190 | 190 | 190 | 190 | 190 | 190 | 190 |
| System Pressure | 1200 | 1200 | 1200 | 1200 | 1200 | 1200 | 1200 | 1240 | 1200 |
| Liq. Feed Rate (ml/hr) | 50 | 50 | 50 | 50 | 50 | 50 | 50 | 50 | 50 |
| Glycerol Feed Concentration | 34.23 | 34.23 | 34.23 | 39.96 | 39.96 | 39.96 | 39.90 | 39.96 | 39.96 |
| (wt %) | | | | | | | | | |
| NaOH Feed Concentration | 2.10 | 2.10 | 2.10 | 2.10 | 2.10 | 2.10 | 2.10 | 2.10 | 2.10 |
| (wt %) | | | | | | | | | |
| H2/Glycerol Molar Feed Ratio | 5 | 5 | 5 | 5 | 5 | 5 | 5 | 5 | 5 |
| H2 Flow Rate (SCCM) | 450 | 450 | 450 | 454 | 454 | 454 | 450 | 450 | 450 |
| % Wt. Recovery | 97.16 | 99.23 | 98.17 | 98.335 | 97.720 | 96.854 | 97.19 | 91.46 | 98.90 |
| % Carbon Recovery | 97.94 | 105.58 | 101.27 | 91.552 | 93.731 | 93.779 | 95.75 | 87.46 | 95.15 |
| Glycerol Conversion | 0.92 | 0.90 | 0.90 | 0.950 | 0.947 | 0.947 | 0.86 | 0.86 | 0.85 |
| (By Difference) | | | | | | | | | |
| Selectivities | | | | | | | | | |
| PG C Molar Selectivity | 0.934 | 0.929 | 0.935 | 0.890 | 0.895 | 0.900 | 0.93 | 0.93 | 0.93 |
| Lactate C Molar Selectivity | 0.018 | 0.016 | 0.017 | 0.034 | 0.030 | 0.029 | 0.02 | 0.02 | 0.02 |
| EG C Molar Selectivity | 0.032 | 0.031 | 0.032 | 0.033 | 0.033 | 0.033 | 0.03 | 0.03 | 0.04 |
| Methanol C molar Selectivity | 0.011 | 0.015 | 0.009 | 0.015 | 0.017 | 0.014 | 0.01 | 0.01 | 0.01 |
| Ethanol C Molar Selectivity | 0.003 | 0.003 | 0.003 | 0.010 | 0.009 | 0.010 | 0.00 | 0.00 | 0.00 |
| Propanol (1&2) C Molar | 0.002 | 0.005 | 0.004 | | | | | | |
| Selectivity | | | | | | | | | |
| LHSV (cc feed/cc cat/h) | 1.67 | 1.67 | 1.67 | | | | | | |
| WHSV (g gly/g cat/h) | 1.51 | 1.51 | 1.52 | | | | | | |
| Space Time Yield | 0.44 | 0.47 | 0.45 | | | | | | |
| (g PG/cc cat/h) | | | | | | | | | |

TABLE 2
Catalyst G

| System Conditions | F65-1 | F65-2 | F65-3 | F65-4 | F65-5 | F65-6 | F65-19 |
|---|---|---|---|---|---|---|---|
| Hours on stream | 23.6 | 47.4 | 143.2 | 167.6 | 191.7 | 240.9 | 744.4 |
| Cat. Bed Temp (° C.) | 190 | 190 | 190 | 190 | 190 | 190 | 190 |
| System Pressure | 1200 | 1200 | 1200 | 1200 | 1200 | 1200 | 1200 |
| Liq. Feed Rate (ml/hr) | 50 | 50 | 50 | 50 | 50 | 50 | 50 |
| Glycerol Feed Concentration (wt %) | 39.96 | 39.96 | 39.96 | 39.96 | 40.20 | 40.20 | 39.30 |
| NaOH Feed Concentration (wt %) | 2.10 | 2.10 | 2.10 | 2.10 | 2.10 | 2.10 | 2.10 |
| H2/Glycerol Molar Feed Ratio | 5 | 5 | 5 | 5 | 5 | 5 | 5 |
| H2 Flow Rate (SCCM) | 454 | 454 | 454 | 454 | 454 | 454 | 454 |
| % Wt. Recovery | 98.335 | 97.720 | 96.854 | 97.674 | 98.621 | 97.218 | 98.500 |
| % Carbon Recovery | 91.552 | 93.731 | 93.779 | 93.774 | 93.417 | 94.184 | 96.352 |
| Glycerol Conversion (By Difference) | 0.950 | 0.947 | 0.947 | 0.946 | 0.946 | 0.944 | 0.851 |
| Selectivities | | | | | | | |
| PG C Molar Selectivity | 0.890 | 0.895 | 0.900 | 0.901 | 0.897 | 0.904 | 0.903 |
| Lactate C Molar Selectivity | 0.034 | 0.030 | 0.029 | 0.027 | 0.026 | 0.026 | 0.027 |
| EG C Molar Selectivity | 0.033 | 0.033 | 0.033 | 0.034 | 0.034 | 0.034 | 0.031 |
| Methanol C molar Selectivity | 0.015 | 0.017 | 0.014 | 0.013 | 0.017 | 0.014 | 0.015 |
| Ethanol C Molar Selectivity | 0.010 | 0.009 | 0.010 | 0.010 | 0.009 | 0.008 | 0.009 |

TABLE 3
Catalysts K, L, and J (UOP L was reduced at 320° C., UOP K was
reduced at 290° C. and UOP J was reduced at 260° C.;
each catalyst was depassivated at 210° C.)

| System Conditions | | | | | | |
|---|---|---|---|---|---|---|
| 2.5% Co + .45% Pd + 2.37% | F72-1 | F72-2 | F74-1 | F74-2 | F83-1 | F83-2 |
| Re on ROX 0.8 | UOP L | | UOP K | | UOP J | |
| Hours on stream | 23:09:00 | 47:23:00 | 24:09:00 | 48:01:00 | 3:47:00 | 27:08:00 |
| Cat. Bed Temp (° C.) | 190 | 190 | 190 | 190 | 190 | 190 |
| System Pressure | 1200 | 1200 | 1200 | 1200 | 1200 | 1200 |
| Liq. Feed Rate (ml/hr) | 50 | 50 | 50 | 50 | 50 | 50 |
| Glycerol Feed Concentration (wt %) | 34.70 | 34.70 | 34.70 | 34.70 | 35.64 | 42.73 |
| NaOH Feed Concentration (wt %) | 2.10 | 2.10 | 2.10 | 2.10 | 2.10 | 2.10 |
| H2/Glycerol Molar Feed Ratio | 5 | 5 | 5 | 5 | 5 | 5 |
| H2 Flow Rate (SCCM) | 450 | 450 | 450 | 450 | 450 | 450 |
| % Wt. Recovery | 97.55 | 96.82 | 98.14 | 98.36 | 97.55 | 101.19 |
| % Carbon Recovery | 93.83 | 96.57 | 98.88 | 98.26 | 97.58 | 99.22 |
| Glycerol Conversion (By Difference) | 0.93 | 0.94 | 0.90 | 0.92 | 0.68 | 0.79 |
| LHSV (cc feed/cc cat/h) | 1.67 | 1.67 | 1.67 | 1.67 | 1.67 | 1.67 |
| WHSV (g gly/g cat/h) | 1.48 | 1.48 | 1.49 | 1.49 | 1.52 | 1.83 |
| Space Time Yield (g PG/cc cat/h) | 0.43 | 0.44 | 0.44 | 0.45 | 0.43 | 0.48 |
| Selectivities | | | | | | |
| PG C Molar Selectivity | 0.922 | 0.915 | 0.930 | 0.927 | 0.929 | 0.926 |
| Lactate C Molar Selectivity | 0.023 | 0.024 | 0.018 | 0.018 | 0.016 | 0.018 |
| EG C Molar Selectivity | 0.031 | 0.031 | 0.030 | 0.032 | 0.030 | 0.031 |
| Methanol C molar Selectivity | 0.010 | 0.012 | 0.015 | 0.013 | 0.014 | 0.011 |
| Ethanol C Molar Selectivity | 0.005 | 0.005 | 0.003 | 0.002 | 0.002 | 0.006 |
| Propanol (1&2) C Molar Selectivity | 0.0021 | 0.0037 | 0.0021 | 0.0011 | 0.006 | 0.001 |

### EXAMPLE 4

### Co—Pd—Re Catalysts

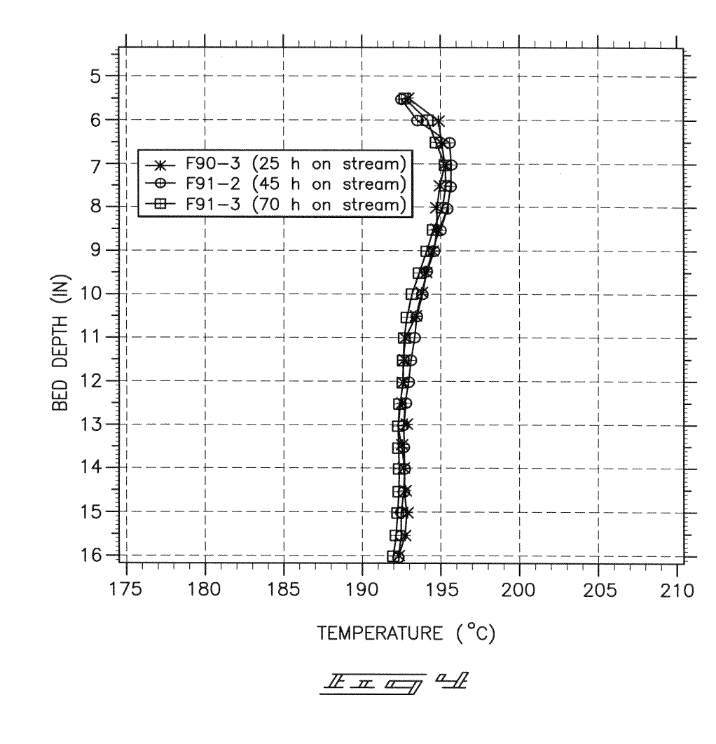

In accordance with the processes described herein two catalysts can be prepared; catalysts H (2.20% Co, 0.47% Pd, 2.39% Re on ROX) and I (2.83% Co, 0.45% Pd, 2.36% Re on ROX) as represented in Table 4 below. Table 4 and FIG. 4 demonstrate the data acquired utilizing these catalysts prepared according to indicated methods.

TABLE 4
Catalyst H and I.

| System Conditions | F90-3 | F91-2 | F91-3 | F93-2 |
|---|---|---|---|---|
| Catalyst | UOP H | UOP H | UOP H | UOP I |
| Reduction | 210° C. | JFW 320° C., 210° C. | | JFW |
| | | | | 320° C., |
| | | | | 210° C. |
| Hours on stream | 25:16:00 | 45:19:00 | 69:57:00 | 24:10:00 |
| Cat. Bed Temp (° C.) | 190 | 190 | 190 | 190 |
| System Pressure | 1200 | 1200 | 1200 | 1200 |
| Liq. Feed Rate (ml/hr) | 50 | 50 | 50 | 50 |
| Glycerol Feed | 34.28 | 34.28 | 34.28 | 34.23 |
| Concentration (wt %) | | | | |
| NaO H Feed | 2.10 | 2.10 | 2.10 | 2.10 |
| Concentration (wt %) | | | | |
| H2/Glycerol Molar | 5 | 5 | 5 | 5 |
| Feed Ratio | | | | |
| H2 Flow Rate (SCCM) | 450 | 450 | 450 | 450 |
| % Wt. Recovery | 98.58 | 98.06 | 97.36 | 96.50 |
| % Carbon Recovery | 104.39 | 98.85 | 99.09 | 102.22 |
| Glycerol Conversion | 0.68 | 0.70 | 0.70 | 0.79 |
| (By Difference) | | | | |
| LHSV (cc feed/cc cat/h) | 1.67 | 1.67 | 1.67 | 1.67 |
| WHSV (g gly/g cat/h) | 1.39 | 1.50 | 1.50 | 1.49 |
| Space Time Yield (g | 0.36 | 0.34 | 0.34 | 0.40 |
| PG/cc cat/h) | | | | |
| Selectivities | | | | |
| PG C Molar Selectivity | 0.942 | 0.947 | 0.943 | 0.940 |
| Lactate C Molar | 0.013 | 0.012 | 0.013 | 0.014 |
| Selectivity | | | | |
| EG C Molar Selectivity | 0.029 | 0.029 | 0.029 | 0.031 |
| Methanol C Molar | 0.010 | 0.008 | 0.010 | 0.015 |
| Selectivity | | | | |
| Ethanol C Molar | 0.002 | 0.003 | 0.003 | 0.000 |
| Selectivity | | | | |
| Propanol (1&2) C Molar | 0.0018 | 0.0006 | 0.0012 | 0.0000 |
| Selectivity | | | | |

Regarding Table 5 below, twelve hydrogenolysis catalysts (2.5% Co, 2.4% Re, 0.45% Pd on Norit ROX 0.8) can be reduced and passivated. The hydrogenolysis of polyhydric alcohol compounds subsequent to catalyst preparation can be performed in a trickle bed reactor experiments in accordance with the parameters detailed below.

TABLE 5
Catalyst Preparation Parameters

| | Max | Hold | | | | | | | |
|---|---|---|---|---|---|---|---|---|---|
| | Temp | Time, | mol % | Inlet | | | | PNNL | |
| Expt # | ° C. | hrs | H2 | Moisture | Ramp | Passivation | nb# | 59260- | Run |
| 1 | 290 | 3 | 15 | 0 | standard | standard | 32895-40 | -36-40 | F114 |
| 2 | 290 | 12 | 15 | 0 | standard | standard | 32895-41 | -36-41 | F115 |
| 3 | 320 | 3 | 15 | 0 | standard | standard | 32895-42 | -36-42 | F102 |
| 4 | 320 | 12 | 15 | 0 | standard | standard | 32895-43 | -36-43 | |
| 5 | 350 | 3 | 15 | 0 | standard | standard | 32895-49 | -36-49 | F117 |
| 6 | 350 | 12 | 15 | 0 | standard | standard | 32895-50 | -36-50 | F113 |
| 7 | 320 | 3 | 5 | 0 | standard | standard | 32895-51 | -36-51 | F122 |
| 8 | 320 | 3 | 50 | 0 | standard | standard | 32895-52 | -36-52 | F126 |
| 9 | 320 | 3 | 15 | 2 mol % | standard | standard | 32895-53 | -36-53 | F106 |
| 10 | 320 | 3 | 15 | 0 | N2 hold at | standard | 32895-54 | -36-54 | F119 |
| | | | | | 350° C., 3 h | | | | |
| 11 | 320 | 3 | 15 | 0 | standard | 250° F. | 32895-55 | -36-55 | F121 |
| | | | | | | exotherm | | | |
| 12 | 320 | 3 | 15 | 0 | standard | standard | 32895-56 | -36-56 | F108 |

Each catalyst can first be reduced and then passivated. As part of the example, a dried 30 cc sample of catalyst containing 2.5% Co, 0.45% Pd and 2.4% Re on Norit ROX 0.8 extrudate can be loaded into a down-flow trickle bed reactor. A 250 sccm gas flow of H₂ can be initiated and the catalyst depassivated by raising the temperature of the reactor 1.5° C./min to 210° C, for example. The temperature can be maintained for 12 h and then cooled over 1 h to 190 ° C. The gas flow rate can then be increased to 450 sccm and the pressure increased to 1200 psig.

Glycerol feedstock (˜40 wt % glycerol, 1.0 wt % NaOH) can be fed to the reactor at a rate of 1.2 LHSV (35 mL/h). In some of the cases, water can be added during the depassivation to simulate water roll-up. Typically 50 ml/h and 35 ml/h samples can be taken.

TABLE 6
Reduction Study

| | Max | Hold | mol | | | | | | |
|---|---|---|---|---|---|---|---|---|---|
| Expt | Temp | Time, | % | Inlet | | | | PNNL | |
| # | ° C. | hrs | H2 | Moisture | Ramp | Passivation | nb# | 59260- | Run |
| 3 | 320 | 3 | 15 | 0 | standard | standard | 32895-42 | -36-42 | F102 |
| 5 | 350 | 3 | 15 | 0 | standard | standard | 32895-49 | -36-49 | F117 |
| 10 | 320 | 3 | 15 | 0 | N2 hold at | standard | 32895-54 | -36-54 | F119 |
| | | | | | 350 C. | | | | |
| | | | | | (3 hrs) | | | | |
| 9 | 320 | 3 | 15 | 2 mol % | standard | standard | 32895-53 | -36-53 | F106 |
| 11 | 320 | 3 | 15 | 0 | standard | 250° F. | 32895-55 | -36-55 | F121 |
| | | | | | | exotherm | | | |
| 12 | 320 | 3 | 15 | 0 | standard | standard | 32895-56 | -36-56 | F108 |
| 8 | 320 | 3 | 50 | 0 | standard | standard | 32895-52 | -36-52 | F126 |
| 4 | 320 | 12 | 15 | 0 | standard | standard | 32895-43 | -36-43 | F127 |
| 1 | 290 | 3 | 15 | 0 | standard | standard | 32895-40 | -36-40 | F114 |
| 2 | 290 | 12 | 15 | 0 | standard | standard | 32895-41 | -36-41 | F115 |
| 7 | 320 | 3 | 5 | 0 | standard | standard | 32895-51 | -36-51 | F122 |
| 6 | 350 | 12 | 15 | 0 | standard | standard | 32895-50 | -36-50 | F113 |

| | 35 ml/h | | 50 ml/h | | | | 35 ml/h | 50 ml/h |
|---|---|---|---|---|---|---|---|---|
| | Gly. | P.G. | Gly. | P.G. | % Conv. | % Sel. | % Yield | % Yield |
| Expt | Conv. | Sel. | Conv. | Sel. | Change | Change | (Conv. × | (Conv. × |
| # | % | % | %^{a} | %^{a} | (35-50 ml/h) | (35-50 ml/h) | Sel.) | Sel.) |
| 3 | 91 | 94 | 72 | 94 | −20.9 | 0.0 | 85.5 | 67.7 |
| 5 | 91 | 94 | — | — | — | — | 85.5 | — |
| 10 | 91 | 93 | 86 | 92 | −5.5 | −1.1 | 84.6 | 79.1 |
| 9 | 90 | 95 | 80 | 94 | −11.1 | −1.1 | 85.5 | 75.2 |
| 11 | 90 | 95 | 83 | 94 | −7.8 | −1.1 | 85.5 | 78.0 |
| 12 | 88 | 94 | 81 | 93 | −8.0 | −1.1 | 82.7 | 75.3 |
| 8 | 87 | 93 | 70 | 92 | −19.5 | −1.1 | 80.9 | 64.4 |
| 4 | 86 | 94 | 82 | 93 | −4.7 | −1.1 | 80.8 | 76.3 |
| 1 | 85 | 95 | — | — | — | — | 80.8 | — |
| 2 | 83 | 94 | — | — | — | — | 78.0 | — |
| 7 | 79 | 95 | 75 | 95 | −5.1 | 0.0 | 75.1 | 71.3 |
| 6 | 73 | 95 | 65 | 94 | −11.0 | −1.1 | 69.4 | 61.1 |

^{a}The 50 ml/h F102 run was done at a hydrogen to glycerol ratio of 3.5 not 5 as all others were.
Notes:
all reductions done using a 1.5° C./min ramp under 15% H₂ unless otherwise noted.
^{a}2 mol % H₂O (mimic water roll-up);
^{b}350° C. N₂ calcination prior to reduction;
^{c}121° C. exotherm during passivation;
^{d}5% H₂;
^{e}50% H2.

TABLE 7
Catalyst at 35 ml/hr feed rate

| | 290° C. | | 350° C. | | 320° C. | |
|---|---|---|---|---|---|---|
| | 3 h | 12 h | 3 h | 12 h | 3 h | 3 h |
| System Conditions | F114-4 | F115-4 | F117-4 | F113-1 | F102-2 | F108-1 |
| Catalyst UOP 32895- | -40 | -41 | -49 | -50 | -42 | -56 |
| 2.5% Co + .45% Pd + 2.37% Re on | | | | | | |
| ROX 0.8 | | | | | | |
| Hours on stream | 42.8 | 81.1 | 24.6 | 62.9 | 498.2 | 66.2 |
| Cat. Bed Temp (° C.) | 190 | 190 | 190 | 190 | 190 | 190 |
| System Pressure | 1200 | 1200 | 1200 | 1200 | 1200 | 1200 |
| Liq. Feed Rate (ml/hr) | 35 | 35 | 35 | 35 | 35 | 35 |
| Glycerol Feed Concentration (wt %) | 36.16 | 39.34 | 39.81 | 34.05 | 38.71 | 40.31 |
| NaOH Feed Concentration (wt %) | 1.00 | 1.00 | 1.00 | 1.00 | 1.00 | 1.00 |
| H2/Glycerol Molar Feed Ratio | 5 | 5 | 5 | 5 | 5 | 5 |
| H2 Flow Rate (SCCM) | 317 | 317 | 317 | 317 | 318 | 318 |
| % Wt. Recovery | 97.71 | 99.06 | 98.61 | 97.48 | 97.49 | 96.57 |
| % Carbon Recovery | 103.48 | 87.44 | 94.52 | 107.49 | 94.51 | 86.23 |
| LHSV (cc feed/cc cat/h) | 1.17 | 117 | 1.17 | 1.17 | 1.17 | 1.17 |
| WHSV (g gly/g cat/h) | 1.08 | 1.18 | 1.21 | 1.01 | 1.15 | 1.20 |
| Space Time Yield (g PG/cc cat/h) | 0.34 | 0.30 | 0.34 | 0.28 | 0.34 | 0.30 |
| Glycerol Conversion (By Difference) | 0.88 | 0.88 | 0.91 | 0.73 | 0.91 | 0.88 |
| Selectivities | | | | | | |
| PG C Molar Selectivity | 0.94 | 0.94 | 0.94 | 0.95 | 0.94 | 0.94 |
| EG C Molar Selectivity | 0.030 | 0.030 | 0.030 | 0.030 | 0.029 | 0.033 |
| Lactate C Molar Selectivity | 0.013 | 0.011 | 0.012 | 0.011 | 0.015 | 0.014 |
| Glycerate C Molar Selectivity | 0.0006 | 0.0005 | 0.0006 | 0.0001 | 0.0004 | 0.0002 |
| Glycolate C Molar Selectivity | 0.0005 | 0.0002 | 0.0007 | 0.0002 | 0.0000 | 0.0002 |
| Formate C Molar Selectivity | 0.0012 | 0.0011 | 0.0025 | 0.0007 | 0.0000 | 0.0000 |
| 1,3 Propanediol C Molar Selectivity | 0.0000 | 0.0000 | 0.0000 | 0.0000 | 0.0000 | 0.0000 |
| Methanol C Molar Selectivity | 0.01 | 0.01 | 0.013 | 0.0103 | 0.008 | 0.010 |
| Ethanol C Molar Selectivity | 0.00 | 0.00 | 0.001 | 0.0009 | 0.003 | 0.001 |
| 1-PropanolC Molar Selectivity | 0.00 | 0.00 | 0.001 | 0.0008 | 0.0000 | 0.0000 |
| 2-Propanol C Molar Selectivity | 0.00 | 0.00 | 0.003 | 0.0009 | 0.0023 | 0.0020 |
| | 320° C. | | | | | |
| | 3 h^{a} | 3 h^{b} | 3 h^{c} | 3 h^{d} | 3 h^{e} | 12 h |
| System Conditions | F106-2 | F119-2 | F121-3 | F122-2 | F126-2 | F127-1 |
| Catalyst UOP 32895- | -53 | -54 | -55 | -51 | -52 | -43 |
| 2.5% Co + .45% Pd + 2.37% Re on | | | | | | |
| ROX 0.8 | | | | | | |
| Hours on stream | 43.1 | 72.1 | 22.0 | 7.7 | 64.2 | 20.2 |
| Cat. Bed Temp (° C.) | 190 | 190 | 190 | 190 | 190 | 190 |
| System Pressure | 1200 | 1200 | 1200 | 1200 | 1200 | 1200 |
| Liq. Feed Rate (ml/hr) | 35 | 35 | 35 | 35 | 35 | 35 |
| Glycerol Feed Concentration (wt %) | 40.25 | 38.01 | 39.43 | 39.42 | 38.33 | 38.90 |
| NaOH Feed Concentration (wt %) | 1.00 | 1.00 | 1.00 | 1.00 | 1.00 | 1.00 |
| H2/Glycerol Molar Feed Ratio | 10 | 5 | 5 | 5 | 5 | 5 |
| H2 Flow Rate (SCCM) | 636 | 317 | 317 | 317 | 317 | 317 |
| % Wt. Recovery | 97.41 | 97.92 | 98.44 | 98.75 | 99.14 | 98.95 |
| % Carbon Recovery | 97.16 | 98.27 | 95.80 | 95.63 | 94.83 | 103.26 |
| LHSV (cc feed/cc cat/h) | 1.17 | 1.17 | 1.17 | 1.17 | 1.17 | 1.17 |
| WHSV (g gly/g cat/h) | 1.19 | 1.15 | 1.11 | 1.20 | 1.12 | 1.16 |
| Space Time Yield (g PG/cc cat/h) | 0.36 | 0.34 | 0.34 | 0.30 | 0.31 | 0.35 |
| Glycerol Conversion (By Difference) | 0.90 | 0.91 | 0.90 | 0.79 | 0.87 | 0.86 |
| Selectivities | | | | | | |
| PG C Molar Selectivity | 0.95 | 0.93 | 0.95 | 0.95 | 0.93 | 0.938 |
| EG C Molar Selectivity | 0.033 | 0.029 | 0.030 | 0.029 | 0.028 | 0.03 |
| Lactate C Molar Selectivity | 0.014 | 0.014 | 0.012 | 0.012 | 0.019 | 0.013 |
| Glycerate C Molar Selectivity | 0.0003 | 0.0004 | 0.0004 | 0.0008 | 0.0005 | 0.001 |
| Glycolate C Molar Selectivity | 0.0000 | 0.0003 | 0.0004 | 0.0004 | 0.0002 | 0.0005 |
| Formate C Molar Selectivity | 0.0000 | 0.0007 | 0.0022 | 0.0009 | 0.0013 | 0.0020 |
| 1,3 Propanediol C Molar Selectivity | 0.0000 | 0.0000 | 0.0000 | 0.0000 | 0.0000 | 0.0010 |
| Methanol C Molar Selectivity | 0.003 | 0.018 | 0.007 | 0.006 | 0.014 | 0.0112 |
| Ethanol C Molar Selectivity | 0.002 | 0.002 | 0.001 | 0.001 | 0.004 | 0.002 |
| 1-PropanolC Molar Selectivity | 0.000 | 0.000 | 0.000 | 0.002 | 0.000 | 0.001 |
| 2-Propanol C Molar Selectivity | 0.0031 | 0.002 | 0.001 | 0.001 | 0.004 | 0.002 |

All reductions done using a 1.5° C./min ramp under 15% H₂ unless otherwise noted.
^{a}2 mol % H₂O (mimic water roll-up);
^{b}350° C. N₂ calcination prior to reduction;
^{c}121° C. exotherm during passivation;
^{d}5% H₂;
^{e}50% H₂

TABLE 8
Catalyst at 50 ml/hr feed rate

| | 290° C. | | 350° C. | | 320° C. | |
|---|---|---|---|---|---|---|
| System Conditions | 3 h | 12 h | 3 h | 12 h | 3 h | 3 h |
| 2.5% Co + .45% Pd + 2.37% Re on ROX 0.8 | F114-4 | F115-4 | F117-4 | F113-3 | F102-10 | F108-2 |
| Catalyst UOP 32895- | -40 | -41 | -49 | -50 | -42 | -56 |
| Hours on stream | | | | 90.1 | 739.2 | 89.3 |
| Cat. Bed Temp (° C.) | | | | 190 | 190 | 190 |
| System Pressure | | | | 1200 | 1200 | 1200 |
| Liq. Feed Rate (ml/hr) | | | | 50 | 50 | 50 |
| Glycerol Feed Concentration (wt %) | | | | 37.42 | 40.25 | 40.31 |
| NaOH Feed Concentration (wt %) | | | | 1.00 | 1.00 | 1.00 |
| H2/Glycerol Molar Feed Ratio | | | | 5 | 3.5 | 5 |
| H2 Flow Rate (SCCM) | | | | 454 | 318 | 454 |
| % Wt. Recovery | | | | 97.53 | 97.00 | 98.20 |
| % Carbon Recovery | | | | 105.34 | 93.76 | 97.52 |
| LHSV (cc feed/cc cat/h) | | | | 1.67 | 1.67 | 1.67 |
| WHSV (g gly/g cat/h) | | | | 1.59 | 1.71 | 1.71 |
| Space Time Yield (g PG/cc cat/h) | | | | 0.38 | 0.38 | 0.45 |
| Glycerol Conversion (By Difference) | | | | 0.65 | 0.717 | 0.807 |
| Selectivities | | | | | | |
| PG C Molar Selectivity | | | | 0.94 | 0.936 | 0.932 |
| EG C Molar Selectivity | | | | 0.029 | 0.031 | 0.034 |
| Lactate C Molar Selectivity | | | | 0.010 | 0.017 | 0.016 |
| Glycerate C Molar Selectivity | | | | 0.0002 | 0.0004 | 0.0003 |
| Glycolate C Molar Selectivity | | | | 0.0000 | 0.0000 | 0.0006 |
| Formate C Molar Selectivity | | | | 0.0005 | 0.0000 | 0.0010 |
| 1,3 Propanediol C Molar Selectivity | | | | 0.0000 | 0.0000 | 0.0020 |
| Methanol C Molar Selectivity | | | | 0.0198 | 0.009 | 0.009 |
| Ethanol C Molar Selectivity | | | | 0.0017 | 0.003 | 0.002 |
| 1-PropanolC Molar Selectivity | | | | 0.0000 | 0.0000 | 0.0000 |
| 2-Propanol C Molar Selectivity | | | | 0.0018 | 0.0034 | 0.0031 |
| | 320° C. | | | | | |
| System Conditions | 3 h^{a} | 3 h^{b} | 3 h^{c} | 3 h^{d} | 3 h^{e} | 12 h |
| 2.5% Co + .45% Pd + 2.37% Re on ROX 0.8 | F106-3 | F119-3 | F121-4 | F122-4 | F126-4 | F127-2 |
| Catalyst UOP 32895- | -53 | -54 | -55 | -51 | -52 | -43 |
| Hours on stream | 66.2 | 96.2 | 51.0 | 49.4 | 117.3 | 43.7 |
| Cat. Bed Temp (° C.) | 190 | 190 | 190 | 190 | 190 | 190 |
| System Pressure | 1200 | 1200 | 1200 | 1200 | 1200 | 1200 |
| Liq. Feed Rate (ml/hr) | 50 | 50 | 50 | 50 | 50 | 50 |
| Glycerol Feed Concentration (wt %) | 40.25 | 38.01 | 39.43 | 39.42 | 38.333 | 38.90 |
| NaOH Feed Concentration (wt %) | 1.00 | 1.00 | 1.00 | 1.00 | 1 | 1.00 |
| H2/Glycerol Molar Feed Ratio | 5 | 5 | 5 | 5 | 5 | 5 |
| H2 Flow Rate (SCCM) | 454 | 454 | 454 | 317 | 454 | 454 |
| % Wt. Recovery | 98.02 | 98.60 | 97.29 | 97.93 | 98.40 | 98.96 |
| % Carbon Recovery | 93.68 | 98.76 | 97.18 | 98.83 | 99.66 | 95.91 |
| LHSV (cc feed/cc cat/h) | 1.67 | 1.67 | 1.67 | 1.67 | 1.67 | 1.67 |
| WHSV (g gly/g cat/h) | 1.70 | 1.64 | 1.58 | 1.71 | 1.60 | 1.65 |
| Space Time Yield (g PG/cc cat/h) | 0.43 | 0.46 | 0.46 | 0.43 | 0.38 | 0.44 |
| Glycerol Conversion (By Difference) | 0.797 | 0.86 | 0.83 | 0.75 | 0.70 | 0.82 |
| Selectivities | | | | | | |
| PG C Molar Selectivity | 0.94 | 0.92 | 0.95 | 0.95 | 0.92 | 0.93 |
| EG C Molar Selectivity | 0.034 | 0.030 | 0.030 | 0.029 | 0.028 | 0.030 |
| Lactate C Molar Selectivity | 0.012 | 0.013 | 0.010 | 0.012 | 0.020 | 0.014 |
| Glycerate C Molar Selectivity | 0.0002 | 0.0004 | 0.0003 | 0.0005 | 0.0005 | 0.000 |
| Glycolate C Molar Selectivity | 0.0000 | 0.0000 | 0.0003 | 0.0005 | 0.0005 | 0.001 |
| Formate C Molar Selectivity | 0.0000 | 0.0000 | 0.0011 | 0.0017 | 0.0038 | 0.003 |
| 1,3 Propanediol C Molar Selectivity | 0.0000 | 0.0000 | 0.0000 | 0.0000 | 0.0008 | 0.000 |
| Methanol C Molar Selectivity | 0.014 | 0.031 | 0.008 | 0.004 | 0.017 | 0.014 |
| Ethanol C Molar Selectivity | 0.000 | 0.002 | 0.002 | 0.001 | 0.006 | 0.004 |
| 1-PropanolC Molar Selectivity | 0.000 | 0.000 | 0.000 | 0.001 | 0.000 | 0.000 |
| 2-Propanol C Molar Selectivity | 0.0022 | 0.002 | 0.002 | 0.001 | 0.005 | 0.003 |

All reductions done using a 1.5° C./min ramp under 15% H₂ unless otherwise noted.
^{a}2 mol % H₂O (mimic water roll-up);
^{b}350° C. N₂ calcination prior to reduction;
^{c}121° C. exotherm during passivation;
^{d}5% H₂;
^{e}50% H_{2.}

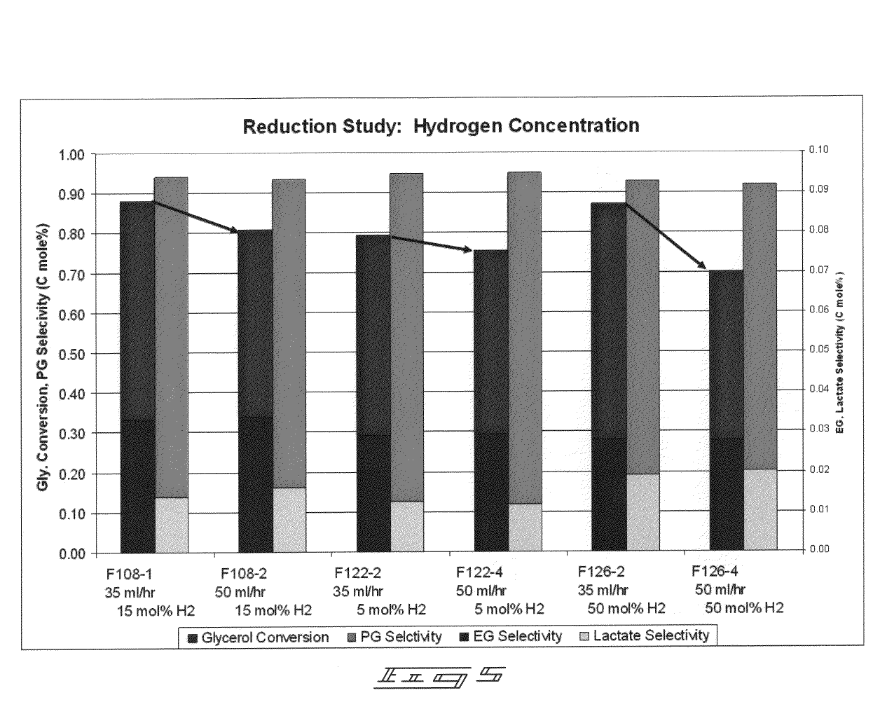

The concentration of the reduction gas at 5, 15, and 50 mol % hydrogen in inert such as N₂ can be varied. In each case an aliquot of catalyst can be reduced 320° C. for 3 hours. Comparing the performance from the series of tests in FIG. 5, the baseline concentration of 15 mol % seems to yield the highest activity for the conditions tested. The results of runs F122 and F126 can indicate that using a 5 mol % or 50 mol % hydrogen concentration during reduction can impact catalyst activity. At the liquid hourly space velocity of 50 ml/hr the performance difference between the baseline 15% and the 50% hydrogen reduction can be nearly 12 percentage points. While 15 mol % hydrogen appears to yield the most active catalyst during reduction, it would not preclude the use of a hydrogen gradient during reduction to further speed the process. Effects of Hydrogen Concentration during Reduction on Performance are depicted graphically in FIG. 5.

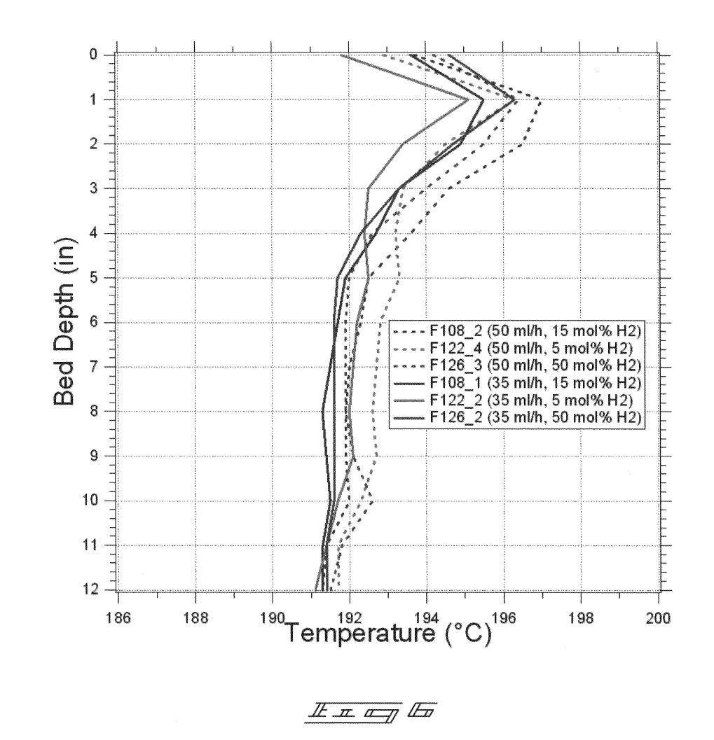

The temperature profiles are shown in FIG. 6. The 5 mol % hydrogen reduction appears to lag behind when the reaction is performed at 35 ml/hr glycerol feed, while the 50 mol % hydrogen reduction tests appear slightly ahead of the 15 mol % run, but nearly equivalent. At a 50 ml/hr feed rate, the bed profile for the 15 mol % appears to peak higher for longer than the 50 mol % test, which appears similar to the 5 mol % test. Reduction Hydrogen Concentration Effect on Reaction Bed Temperature Profile is shown graphically in FIG. 6.

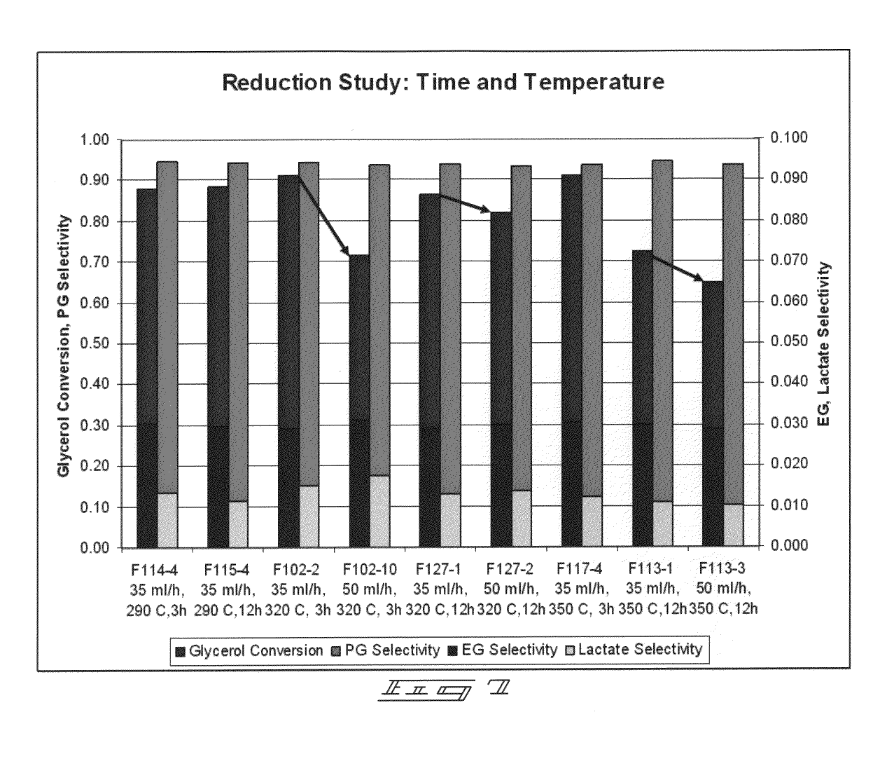

Temperature and duration of the catalyst preparation hold time can be varied on catalysts that all were reduced under 15 mol % hydrogen in inert. Each of these tests can be performed at baseline conditions at 35 ml/hr glycerol feedstock, while some can also performed at 50 ml/hr. Results from the test can be shown in FIG. 7. Again the primary differences between catalyst performances were expressed in the conversion of glycerol only. Selectivity to propylene glycol appeared mostly insensitive to the various reduction condition tests. Effects of Reduction Temperature and Duration on Performance are shown graphically in FIG. 7.

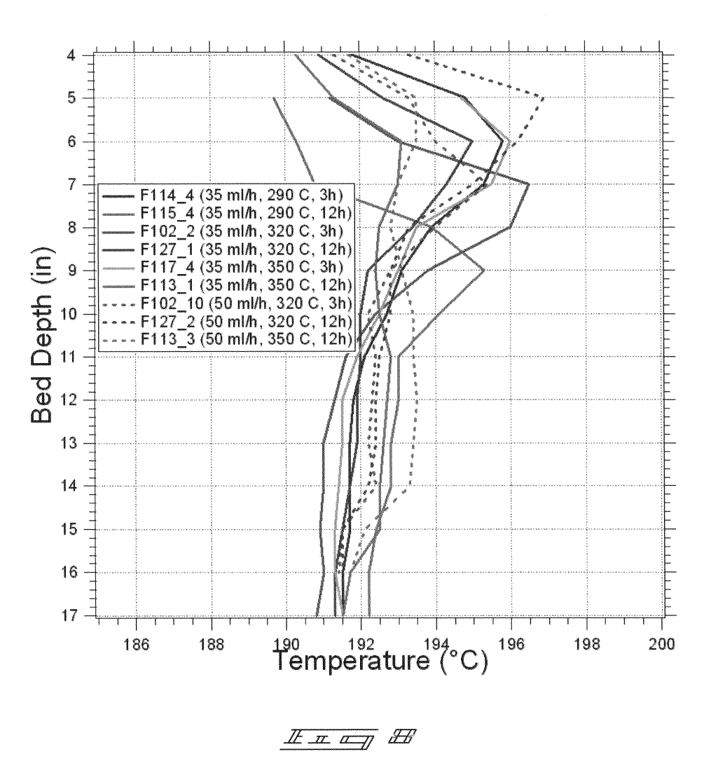

Bed temperature profiles for these tests are shown in FIG. 8. These appear to trend well against the glycerol conversion data and analysis. The variations in the apparent location of the exotherm are due to differences in the location of the catalyst bed in the reactor, rather than changes in activity. Effects of Reduction Temperature and Duration on Bed Temperature Profile are shown graphically in FIG. 8.

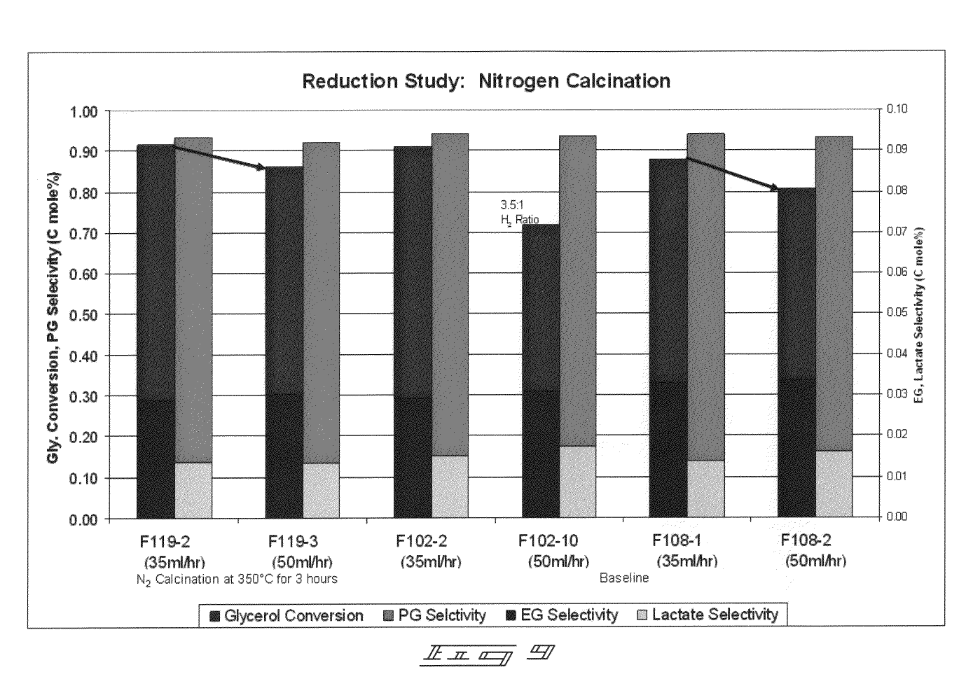

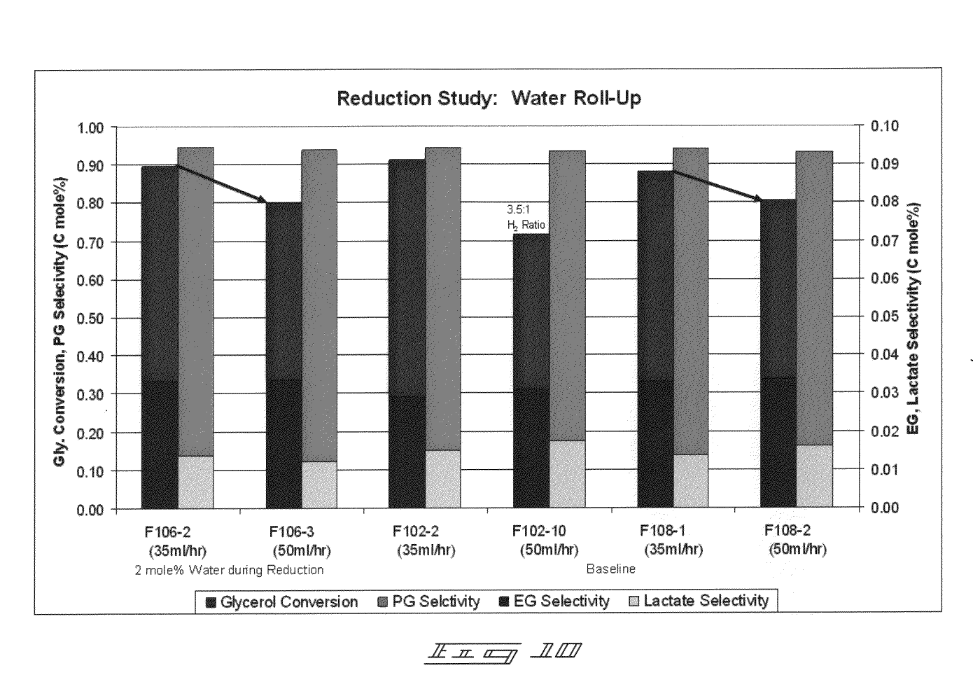

Preparations can also be prepared at 320° C., for 3 h, with 15 mol % hydrogen for the preparation of the 2.5% Co, 0.45% Pd and 2.4% Re catalyst. Effect of Nitrogen Calcination on Performance is shown graphically in FIG. 9. Effect of Water Roll-Up (simulated) During Reduction on Performance is shown graphically in FIG. 10.

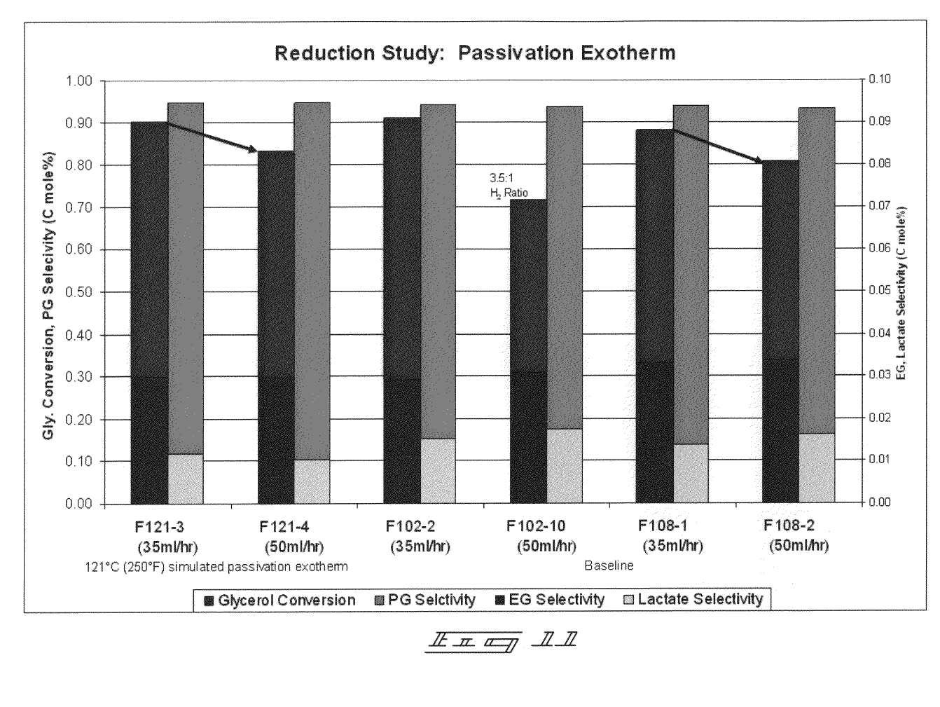

A catalyst can be subjected to a 121° C. (250° F.) simulated exotherm during the passivation process. The passivation exotherm can be the only difference between the baseline catalyst preparation and handling. Effect of Passivation Exotherm (simulated) on Performance is shown graphically in FIG. 11.

In compliance with the statute, embodiments of the invention have been described in language more or less specific as to structural and methodical features. It is to be understood, however, that the entire invention is not limited to the specific features and/or embodiments shown and/or described, since the disclosed embodiments comprise forms of putting the invention into effect. The invention is, therefore, claimed in any of its forms or modifications within the proper scope of the appended claims appropriately interpreted in accordance with the doctrine of equivalents.

1. A hydrogenolysis process comprising:
providing a hydrogenolysis reactor having a catalyst therein, wherein the catalyst comprises Re and one or both of Co and Pd;
exposing the catalyst to a reducing agent in the absence of polyhydric alcohol compound while maintaining a temperature of the catalyst above 290° C.; and
contacting the catalyst with the polyhydric alcohol compound.

2. The process of claim 1 wherein the catalyst comprises Re, Co, and Pd.

3. The process of claim 1 wherein the temperature of the catalyst is maintained between 290° C. and 350° C.

4. The process of claim 1 wherein the temperature of the catalyst is maintained between 290° C. and 320° C.

5. The process of claim 1 wherein the exposing comprises elevating the temperature from a first temperature to at least about 290° C. at a rate of less than about 1.5° C./min, wherein the first temperature is less than 290° C.

6. The process of claim 1 wherein the temperature is maintained for less than about 12 hours.

7. The process of claim 1 wherein the temperature is maintained for from about 3 hours to about 12 hours.

8. The process of claim 1 wherein the reducing atmosphere comprises H₂ and an inert diluent.

9. The process of claim 1 wherein the temperature of the catalyst is maintained above 320° C.

10. The process of claim 9 wherein the reducing atmosphere comprises at least about 4% H₂.

11. A hydrogenolysis process comprising:
providing a passivated catalyst to within a reactor;
exposing the catalyst to a reducing atmosphere while maintaining the catalyst at a temperature less than 210° C.; and
contacting the catalyst with the polyhydric alcohol compound.

12. The process of claim 11 wherein the catalyst comprises one or more of Ni, Re, Co, and Pd.

13. The process of claim 12 wherein the catalyst comprises at least about 5% Ni.

14. The process of claim 12 wherein the catalyst comprises at least about 0.7% Re.

15. The process of claim 14 wherein the catalyst comprises from between about 0.7% and about 2.4% Re.

16. The process of claim 2 wherein the exposing comprises elevating the temperature from a first temperature to at least about 210° C. at a rate of less than about 1.5° C./min, wherein the first temperature is less than 210° C.

17. A hydrogenolysis catalyst preparation method comprising:
exposing the catalyst to a first reducing atmosphere while maintaining the catalyst at a first temperature to reduce at least a portion of the catalyst, wherein the first temperature is the greatest temperature of the catalyst during the exposing;
passivating at least the portion of the catalyst; and
depassivating the portion of the catalyst in the presence of a second reducing atmosphere while maintaining the portion of the catalyst at a second temperature less than the first temperature.

18. The method of claim 17 wherein the first temperature is between about 265° C. and about 350° C.

19. The method of claim 17 wherein the catalyst comprises one or both of Ni and Re.

20. The method of claim 17 wherein the first temperature is greater than about 320° C.

21. The method of claim 20 wherein the second temperature is less than about 320° C.

22. The method of claim 21 wherein the depassivating comprises elevating the catalyst temperature at a rate less than about 2° C./min to the second temperature.

23. The method of claim 21 wherein the depassivating comprises elevating the catalyst temperature at a rate less than about 1.5° C./min to the second temperature.

24. The method of claim 21 wherein during the second reducing atmosphere comprises both H₂ and N₂.

25. The method of claim 24 wherein the second reducing atmosphere is at least 5% H₂.

26. The method of claim 24 wherein the second reducing atmosphere is less than 50% H₂.

27. The method of claim 24 wherein the second reducing atmosphere is from about 5% to about 50% H₂.

28. The method of claim 24 wherein the second reducing atmosphere is from about 15% to about 50% H₂.

29. The method of claim 17 wherein the catalyst comprises one or both of Co, Pd, and Re.

30. The method of claim 29 wherein the first temperature is greater than about 320° C.

31. The method of claim 29 wherein the second temperature is less than about 210° C.

32. The method of claim 30 wherein the depassivating comprises elevating the catalyst temperature at a rate less than about 1.5° C./min to the second temperature.

33. A hydrogenolysis catalyst preparation method comprising:
providing a Co/Pd/Re or Ni/Re hydrogenolysis catalyst; and
maintaining the catalyst at a temperature of at least about 280° C. in the presence of a continuous supply of inert atmosphere.

34. The method of claim 33 wherein the catalyst comprises Re and one or more of Ni, Co, and Pd.

35. The method of claim 33 wherein the temperature is at least about 350° C. and is maintained for at least about 3 hours.

36. The method of claim 33 wherein the inert atmosphere comprises N₂.

37. The method of claim 33 further comprising after maintaining the catalyst, exposing the catalyst to a reducing agent to activate the catalyst.
